(19) United States
(12) Patent Application Publication
(10) Pub. No.: US 2024/0325104 A1
(43) Pub. Date: Oct. 3, 2024

(54) ROBOTIC ARM FOR SUPPORTING A SURGICAL INSTRUMENT

(71) Applicant: HIWIN TECHNOLOGIES CORP., TAICHUNG CITY (TW)

(72) Inventor: Zi-Wei KUO, TAICHUNG CITY (TW)

(21) Appl. No.: 18/192,292

(22) Filed: Mar. 29, 2023

(51) Int. Cl. A61B 34/00

(52) U.S. Cl. CPC A61B 34/74

(57) ABSTRACT

A robotic arm includes a first transmission module, a second transmission module, and a third transmission module. The first transmission module provides a first driven gear to connect with the second transmission module and provides a first fixed gear to engage with a first planetary gear of the second transmission module. When the second transmission module is driven by the first driven gear, the first planetary gear is rotated and revolved around the first fixed gear. The second transmission module uses a second driven gear to connect with the third transmission module connected with a surgical instrument. When driven by the second driven gear, the third transmission module drives the surgical instrument to act. As such, the robotic arm of the present invention is only equipped with a single actuator to achieve an effect of multi-arm linkage.

### BACKGROUND OF THE INVENTION

### 1. Field of the Invention

The present invention relates to robotic arms and more particularly, to a robotic arm for supporting a surgical instrument that has an effect of multi-arm linkage.

### 2. Description of the Related Art

JP 6970780 B2 discloses a medical observation device. An actuator is used in an arm unit to transmit power to a drive shaft, and then the drive shaft transmits power to a joint unit through the engagement of the first and second bevel gears, such that the joint unit can be actuated correspondly. However, in the aforesaid prior art, if the effect of multi-arm linkage would like to be achieved, one actuator needs to be configured in each of the arm units. This causes an increase in cost.

U.S. Pat. No. 11,173,003 B2 discloses a medical system. An input bevel gear is used to drive an input pinion to rotate and revolve around the input bevel gear. During the rotation of the input pinion, an output pinion is driven by a linkage mechanism to rotate and revolve around an output bevel gear. As such, the output bevel gear can be driven by the output pinion to rotate an end support portion synchronously. However, in the aforesaid prior art, the linkage effect of a single joint can be achieved. In addition, the input bevel gear and the output bevel gear are set at two opposite sides of the linkage mechanism, so the volume of the overall structure becomes larger to affect the wire configuration.

### SUMMARY OF THE INVENTION

It is a primary objective of the present invention to provide a robotic arm for supporting a surgical instrument, which can be equipped with a single actuator to achieve an effect of multi-arm linkage.

To attain the above objective, the robotic arm of the present invention is used for retaining a surgical instrument to revolve about a remote center of motion (RCM), comprising a fixed base, a first transmission module, a second transmission module, and a third transmission module. The first transmission module includes a first arm, a first actuator, a first driving gear, a first driven gear, a first fixed shaft, and a first fixed gear. The first arm has a rear end thereof rotatably connected to the fixed base. The first actuator is disposed in the first arm. The first driving gear is disposed in the first arm and connected with the first actuator, such that the first driving gear is driven by the first actuator to rotate. The first driven gear is rotatably connected to a front end of the first arm and engaged with the first driving gear, such that the first driven gear is driven by the first driving gear to rotate. The first fixed shaft is penetrated through the front end of the first arm and the first driven gear, and has one end thereof fixed to the first arm and the other end thereof protruding out of the first arm and connected with the first fixed gear. The second transmission module includes a second arm, a first planetary gear, a first transmission shaft, a second driving gear, and a second driven gear. The second arm has a bottom end thereof receiving the first fixed gear and connected with the first driven gear, such that the second arm is driven by the first driven gear to rotate. The first planetary gear is rotatably connected with the bottom end of the second arm and engaged with the first fixed gear, such that the first planetary gear is driven by the second arm to rotate and revolve around the first fixed gear. The first transmission shaft is disposed in the second arm and has two ends thereof connected with the planetary gear and the second driving gear, such that the first transmission shaft is driven by the first planetary gear to drive the second driving gear to rotate. The second driven gear is rotatably connected with a top end of the second arm and engaged with the second driving gear, such that the second driven gear is driven by the second driving gear to rotate. The third transmission module is connected with the second driven gear of the second transmission module and the surgical instrument, such that the third transmission module is driven by the second driven gear of the second transmission module to retain the surgical instrument to revolve about the remote center of motion (RCM).

It can be seen from the above that when the first actuator is actuated, the second transmission module is driven by the first driven gear to rotate relative to the first transmission module. During rotation of the second transmission module, the first planetary gear is driven by the second transmission module to rotate and revolve around the first fixed gear. In addition, the third transmission module is driven by the second driven gear to drive the surgical instrument to operate. As such, the robotic arm of the present invention is only equipped with a single first actuator to achieve an effect of multi-arm linkage.

Preferably, the second transmission module further includes a second fixed shaft and a second fixed gear. The second fixed shaft is penetrated through the top end of the second arm and the second driven gear, and having one end thereof fixed to the second arm and the other end thereof protruding out of the second arm and connected with the second fixed gear. The third transmission module includes a third arm, a second planetary gear, a second transmission shaft, a third driving gear, and a third driven gear. The third arm has a rear end thereof receiving the second fixed gear and connected with the second driven gear, such that the third arm is driven by the second driven gear to rotate. The second planetary gear is rotatably connected with the rear end of the third arm and engaged with the second fixed gear, such that the second planetary gear is driven by the third arm to rotate and revolve around the second fixed gear. The second transmission shaft is disposed in the third arm and has two ends thereof connected with the second planetary gear and the third driving gear, such that the second transmission shaft is driven by the second planetary gear to drive the third driving gear to rotate. The third driven gear is rotatably connected with a front end of the third arm and connected with the surgical instrument and engaged with the third driving gear, such that the third driven gear is driven by the third driving gear to drive the surgical instrument to revolve about the remote center of motion (RCM).

Preferably, a laser emitter is disposed in the first arm to emit a laser beam towards the remote center of motion for assisting the surgical instrument to be quickly positioned when the surgical instrument is set up.

Preferably, the first transmission module is rotatable relative to the fixed base around a first axial direction coaxial to the laser beam.

Preferably, an angle between the first transmission module and the third transmission module is between 4-8 degrees. An angle between the surgical instrument and the second transmission module is between 62-66 degrees. In this way, if the aforesaid angle exceeds the range, the robotic arm of the present invention cannot meet the conditions for clinical use.

Preferably, a gear ratio between the first fixed gear and the first planetary gear is the same as a gear ratio between the second driving gear and the second driven gear. A gear ratio between the second fixed gear and the second planetary gear is the same as a gear ratio between the third driving gear and the third driven gear. In this way, the second and third arms can be swung synchronously and equiangularly to allow the surgical instrument to be revolved about the remote center of motion (RCM).

Preferably, the first transmission module and the third transmission module are located at the same side of the second transmission module to reduce overall volume.

Preferably, the first fixed shaft has a first wireway coaxially communicating with a first wire hole of the first fixed gear. Further, the second fixed shaft has a second wireway coaxially communicating with a second wire hole of the second fixed gear. In this way, an effect of convenient wiring can be achieved.

Preferably, the front end of the first arm has a first bearing portion and a first threaded hole provided at the first bearing portion. The first fixed shaft has a first lower positioning hole radially communicating with the first wireway. The first transmission module further includes a first positioning plate and a first bolt. The first positioning plate pressed against the first bearing portion of the first arm and the first fixed shaft and has a first upper positioning hole. The first bolt is penetrated through the first upper and lower positioning holes and screwed to the first threaded hole for securing the first fixed shaft and the first arm together. The top end of the second arm has a second bearing portion and a second threaded hole provided at the second bearing portion. The second fixed shaft has a second lower positioning hole radially communicating with the second wireway. The second transmission module further includes a second positioning plate and a second bolt. The second positioning plate presses against the second bearing portion of the second arm and the second fixed shaft and has a second upper positioning hole. The second bolt is penetrated through the second upper and lower positioning holes and screwed to the second threaded hole for securing the second fixed shaft and the second arm together.

Preferably, the second transmission module is rotatable relative to the first transmission module around a second axial direction offset upwards relative to the first axial direction. In this way, more space can be provided below the first transmission module to prevent the first transmission module from collision with a patient's abdomen or other surgical instruments during operation.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a perspective view of a robotic arm of the present invention.

FIG. 2 is a partially exploded view of a first transmission module provided by the robotic arm of the present invention.

FIG. 3 is a partially exploded view of a second transmission module provided by the robotic arm of the present invention.

FIG. 4 is a partially perspective sectional view of the robotic arm of the present invention, showing that the engagement between the first driving gear, the first driven gear, the first fixed gear, and the first planetary gear.

FIG. 5 is a partially sectional view of the robotic arm of the present invention, showing that the engagement between the first driving gear, the first driven gear, the first fixed gear, and the first planetary gear.

FIG. 6 is a partially exploded view of a third transmission module provided by the robotic arm of the present invention.

FIG. 7 is a partially perspective sectional view of the robotic arm of the present invention, showing that the engagement between the second driving gear, the second driven gear, the second fixed gear, and the second planetary gear.

FIG. 8 is a partially sectional view of the robotic arm of the present invention, showing that the engagement between the second driving gear, the second driven gear, the second fixed gear, and the second planetary gear.

FIG. 9 is a lateral view of a fourth transmission module provided by the robotic arm of the present invention, in which a part of the fourth housing is omitted.

FIG. 10 is a partially perspective sectional view of the robotic arm of the present invention, showing that the structural relationship between the third transmission module and the fourth transmission module.

FIG. 11 is a lateral view of the robotic arm of the present invention, showing that the robotic arm is in an initial state.

FIG. 12 is a lateral view of the robotic arm of the present invention, showing that the robotic arm is in an extremely-stretched state.

### DETAILED DESCRIPTION OF THE INVENTION

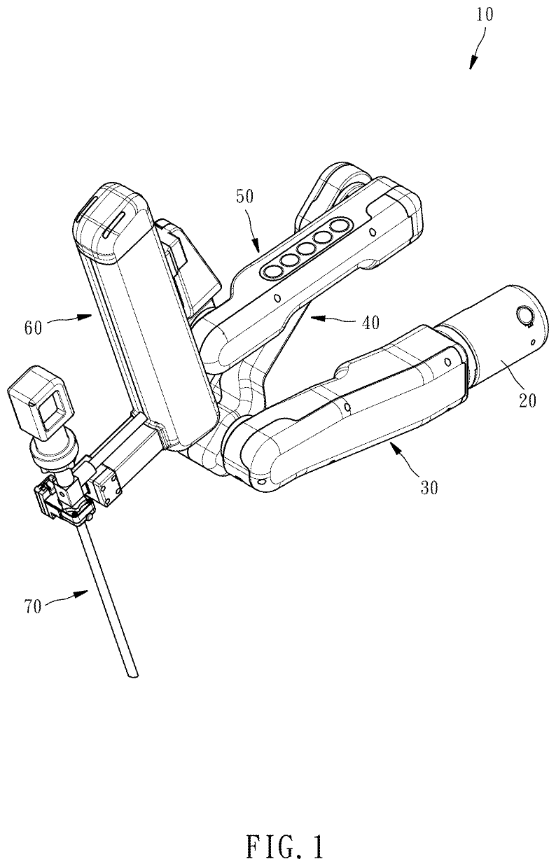

Referring to FIG. 1, a robotic arm 10 of the present invention comprises a fixed base 20, a first transmission module 30, a second transmission module 40, a third transmission module 50, and a fourth transmission module 60.

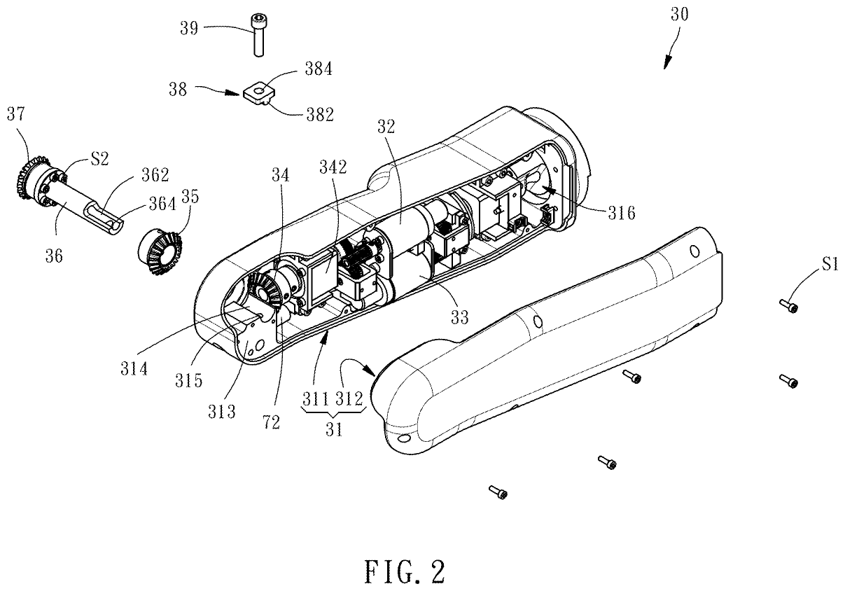

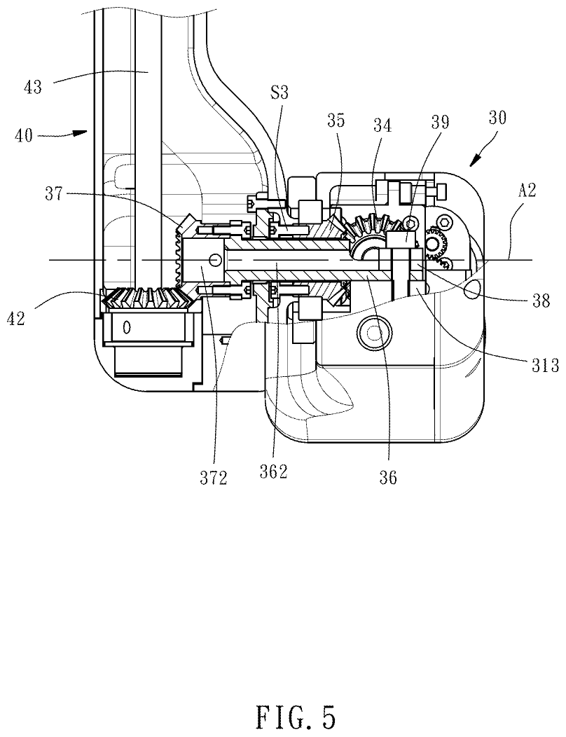

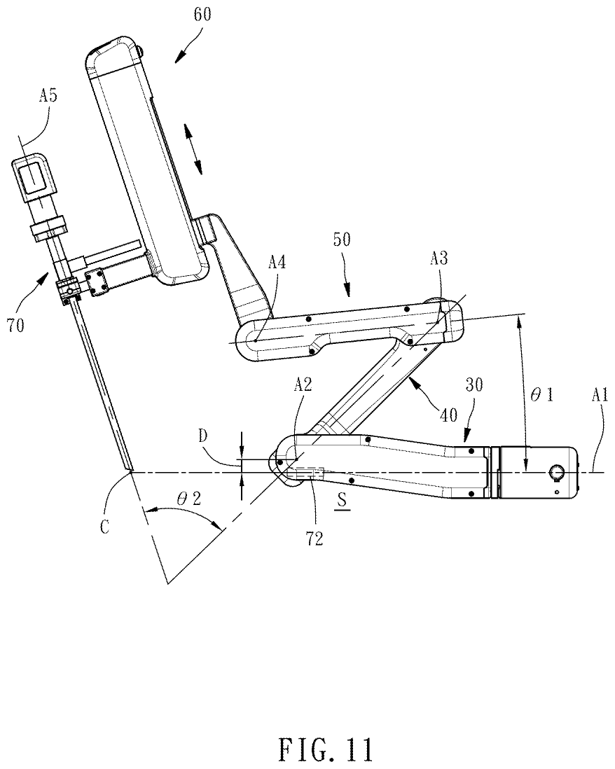

As shown in FIG. 2, the first transmission module 30 includes a first arm 31, a first actuator 32, a second actuator 33, a first driving gear 34, a first driven gear 35, a first fixed shaft 36, a first fixed gear 37, a first positioning plate 38, and a first bolt 39. The first arm 31 has a first housing 311 and a first lateral cover 312 secured to the first housing 311 by using a plurality of screws S1 (the number is unlimited). In addition, the front end of the first housing 311 has a first bearing portion 313 provided with a first bearing groove 314 and a first threaded hole 315 communicating with the first bearing groove 314. The rear end of the first arm 31 is rotatably connected with the fixed base 20 through a joint member 316. The first and second actuators 32, 33 are installed in the first arm 31 and arranged in side-by-side and reverse manner, wherein the second actuator 33 is connected with the joint member 316 to drive the joint member 316 to rotate, such that the first transmission module 30 is rotatable relative to the fixed base 20 around a first axial direction A1 (as shown in FIG. 11). The first driving gear 34 is disposed in the first arm 31 and connected with the first actuator 32 through a reducer 342, such that the first driving gear 34 is driven by the first actuator 32 to rotate. The first driven gear 35 is rotatably connected with the front end of the first housing 311 and engaged with the first driving gear 34, such that the first driven gear 35 is driven by the first driving gear 34 to rotate. As shown in FIGS. 2, 4, and 5, the first fixed shaft 36 is penetrated through the front end of the first housing 311 and the first driven gear 35. One end of the first fixed shaft 36 rests on the first bearing groove 314 of the first bearing portion 313, and the other end of the first fixed shaft 36 protrudes out of the first housing 311 and is connected with the first fixed gear 37 by using a plurality of screws S2 (the number is unlimited). Further, the first fixed shaft 36 has a first wireway 362 through the left and right ends thereof and a first lower positioning hole 364 communicating with the first wireway 362. The first fixed gear 37 has a first wire hole 372 coaxially communicating with the first wireway 362 of the first fixed shaft 36 (as shown in FIG. 5). The first wireway 362 and the first wire hole 372 are provided for penetration of related electrical wires to achieve an effect of convenient wiring. The first positioning plate 38 is positioned in the first wireway 362 of the first fixed shaft 36 through a first positioning protrusion 382, and the first positioning plate 38 has a first upper positioning hole 384 axially communicating with the first lower positioning hole 364. The first bolt 39 is penetrated through the first upper and lower positioning holes 384, 364 and screwed to the first threaded hole 315 for securing the first fixed shaft 36 and the first arm 31 together.

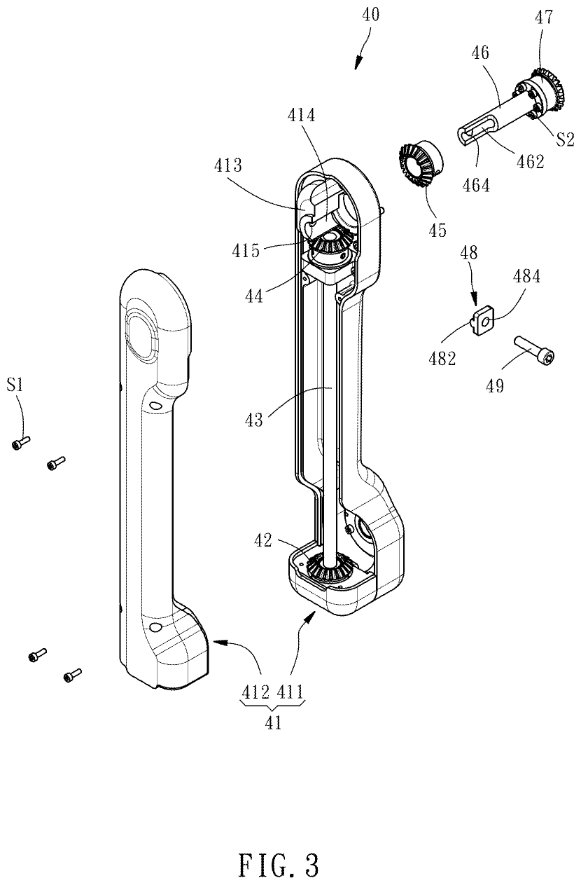

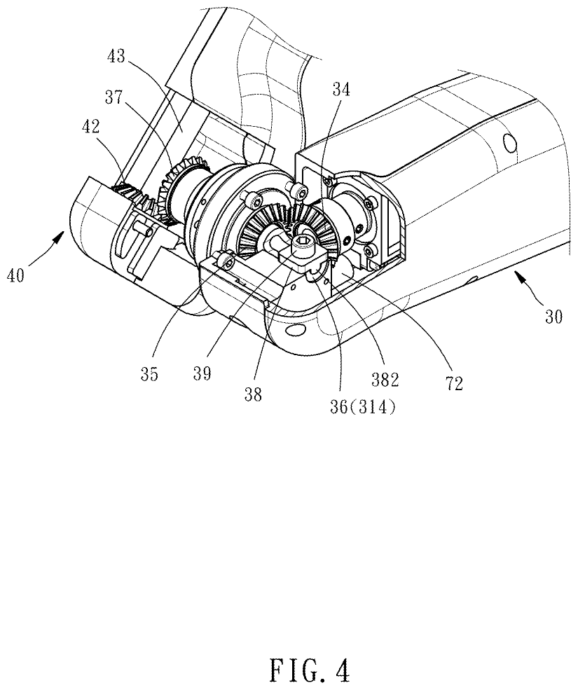

As shown in FIGS. 3 and 4, the second transmission module 40 includes a second arm 41, a first planetary gear 42, a first transmission shaft 43, a second driving gear 44, a second driven gear 45, a second fixed shaft 46, a second fixed gear 47, a second positioning plate 48, and a second bolt 49. The second arm 41 has a second housing 411 and a second lateral cover 412. The top end of the second housing 411 has a second bearing portion 413 provided with a second bearing groove 414 and a second threaded hole 415 communicating with the second bearing groove 414. The bottom end of the second housing 411 receives the first fixed gear 37. The second lateral cover 412 is secured to the second housing 411 by using a plurality of screws S1 (the number is unlimited), and connected with the first driven gear 35 by using a plurality of screws S3 (the number is unlimited), such that the second arm 41 is driven by the first driven gear 35 to rotate relative to the first transmission module 30 around a second axial direction A2 (as shown in FIGS. 5 and 11). The first planetary gear 42 is rotatably connected with the bottom end of the second arm 41 and engaged with the first fixed gear 37, such that the first planetary gear 42 is driven by the second arm 41 to rotate and revolve around the first fixed gear 37. The first transmission shaft 43 is disposed in the second arm 41 and has two ends thereof connected with the first planetary gear 42 and the second driving gear 44, such that the first transmission shaft 43 is driven by the first planetary gear 42 to drive the second driving gear 44 to rotate. The second driven gear 45 is rotatably connected with the top end of the second arm 41 and engaged with the second driving gear 44, such that the second driven gear 45 is driven by the second driving gear 44 to rotate. The second fixed shaft 46 is penetrated through the top end of the second arm 41 and the second driven arm 45. One end of the second fixed shaft 46 rests on the second bearing groove 414 of the second bearing portion 413 of the second arm 41, and the other end of the second fixed shaft 46 protrudes out of the second arm 41 and uses a plurality of screws S2 (the number is unlimited) to connect with the second fixed gear 47. Further, the second fixed shaft 46 has a second wireway 462 through the left and right ends thereof and a second lower positioning hole 464 communicating with the second wireway 462. The second fixed gear 47 has a second wire hole 472 coaxially communicating with the second wireway 462 of the second fixed shaft 46. The second wireway 462 and the second wire hole 472 are provided for penetration of related electrical wires to achieve an effect of convenient wiring. The second positioning plate 48 is positioned in the second wireway 462 of the second fixed shaft 46 through a second positioning protrusion 482, and the second positioning plate 48 has a second upper positioning hole 484 axially communicating with the second lower positioning hole 464. The second bolt 49 is penetrated through the second upper and lower positioning holes 484, 464 and screwed to the second threaded hole 415 for securing the second fixed shaft 46 and the second arm 41 together.

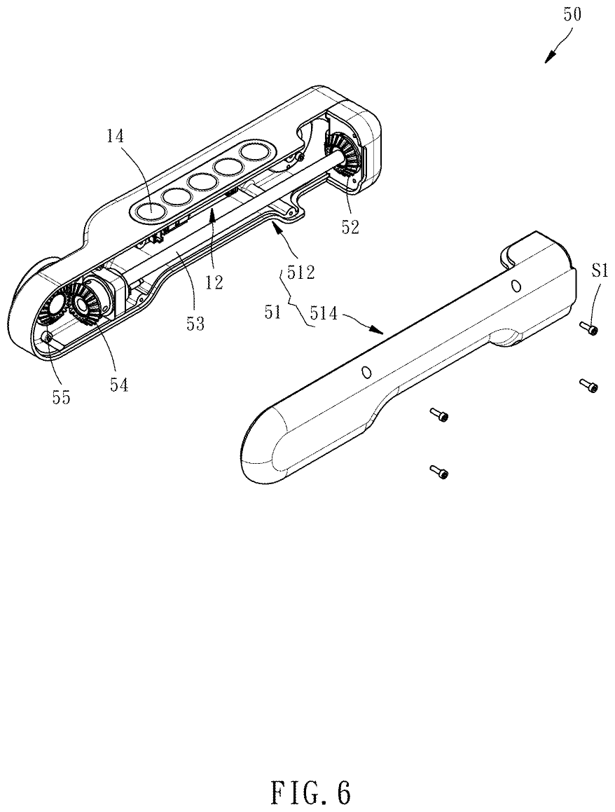

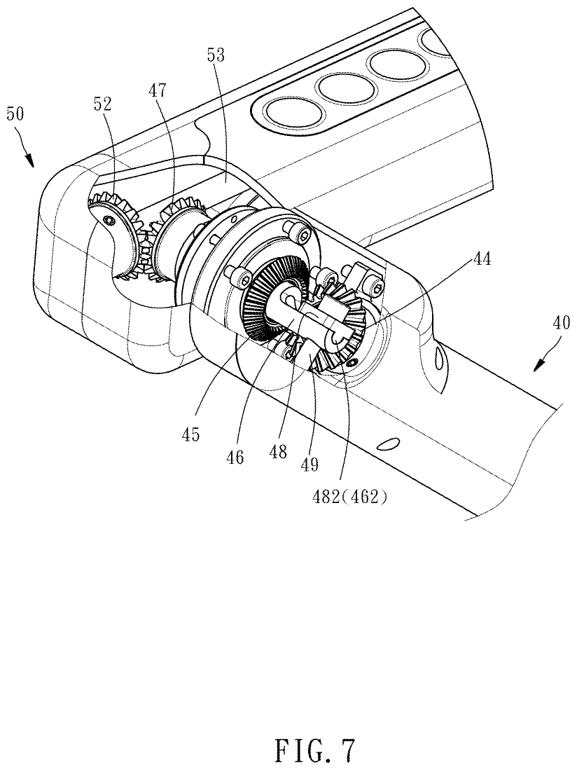

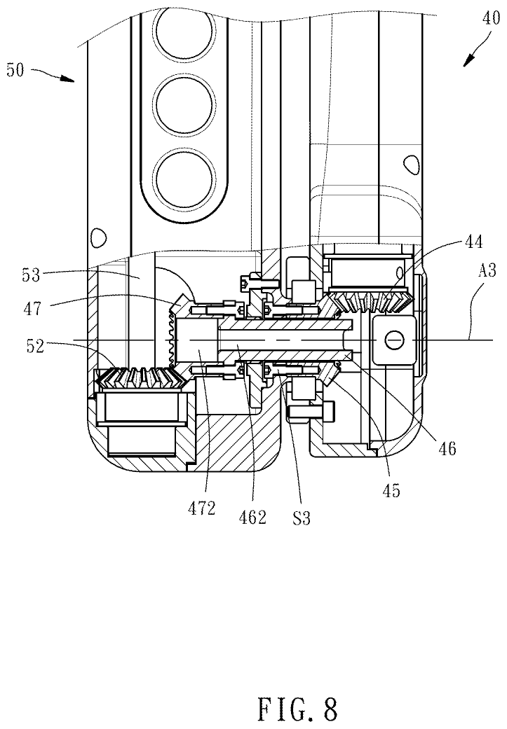

As shown in FIGS. 6-8, the third transmission module 50 includes a third arm 51, a second planetary gear 52, a second transmission shaft 53, a third driving gear 54, and a third driven gear 55. The third arm 51 has a third housing 512 and a third lateral cover 514. The rear end of the third housing 512 is connected with the second driven gear 45 by using a plurality of screws S3 (the number is unlimited), and the rear end of the third housing 512 receives the second fixed gear 47 (as shown in FIG. 7). The third lateral cover 514 is secured to the third housing 512 by using a plurality of screws S1 (the number is unlimited), such that the third arm 51 is driven by the second driven gear 45 to rotate relative to the second transmission module 40 around a third axial direction A3 (as shown in FIGS. 8 and 11). The second planetary gear 52 is rotatably connected with the rear end of the third housing 512 and engaged with the second fixed gear 47, such that the second planetary gear 52 is driven by the third arm 51 to rotate and revolve around the second fixed gear 47. The second transmission shaft 53 is disposed in the third housing 512 and has two ends thereof connected with the second planetary gear 52 and the third driving gear 54, such that the second transmission shaft 53 is driven by the second planetary gear 52 to drive the third driving gear 54 to rotate. The third driven gear 55 is rotatably connected with the front end of the third housing 512 and engaged with the third driving gear 54, such that the third driven gear 55 is driven by the third driving gear 54 to rotate.

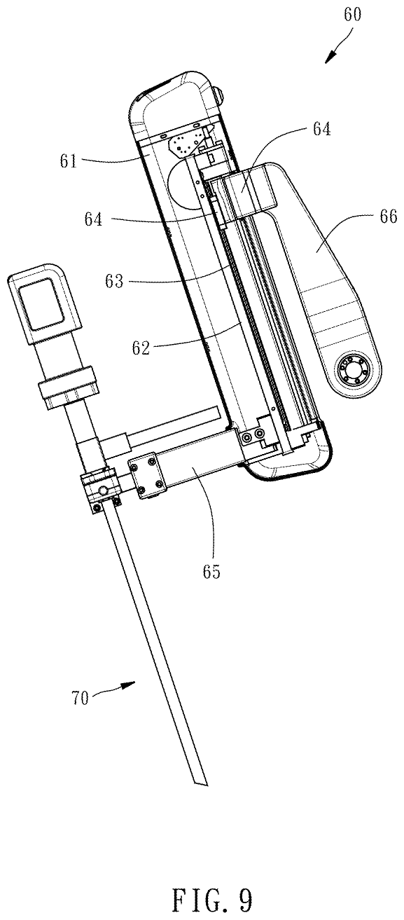

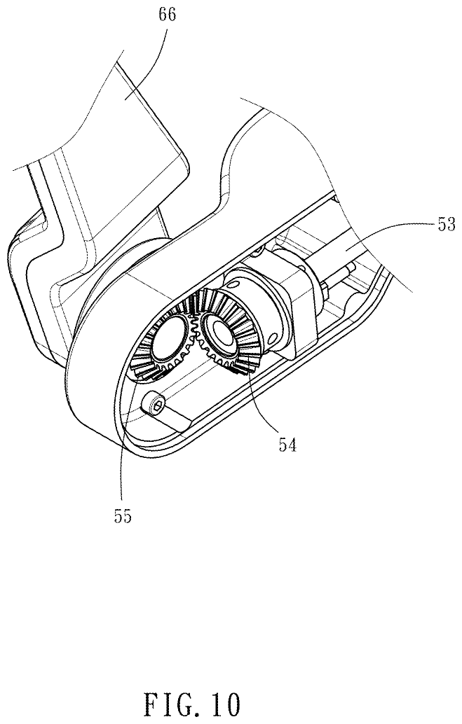

As shown in FIG. 9, the fourth transmission module 60 includes a fourth arm 61, a rail base 62, a rail 63, a slide 64, a front support base 65, and a rear support base 66. The rail base 62 is fixed in the fourth arm 61. The rail 63 is attached to the rear side of the rail base 62. The slide 64 is movably disposed upwards and downwards on the rail 63. The rear end of the front support base 65 is connected with the front side of the rail base 62, and the front end of the front support base 65 is connected with a surgical instrument 70 (here, an endoscope is taken as an example, but not limited to this). The top end of the rear support base 66 is connected with the slide 64, and the bottom end of the rear support base 66 is connected with the third driven gear 55 (as shown in FIG. 10). As such, as shown in FIG. 11, the fourth transmission module 60 is driven by the third driven gear 55 to drive the surgical instrument 70 to rotate relative to the third transmission module 50 around a fourth axial direction A4. Further, the surgical instrument 70 can be moved up and down along a fifth axial direction A5 together with the fourth arm 61 through the slide 64 for adjusting its position.

It is worth mentioning that a gear ratio (r1) between the first fixed gear 37 and the first planetary gear 42 is the same as a gear ratio (r2) between the second driving gear 44 and the second driven gear 45, i.e., r1=r2. A gear ratio (r3) between the second fixed gear 47 and the second planetary gear 52 is the same as a gear ratio (r4) between the third driving gear 54 and the third driven gear 55, i.e., r3-r4. In this way, the second arm 41, the third arm 51, and the fourth arm 61 can be swung synchronously and equiangularly.

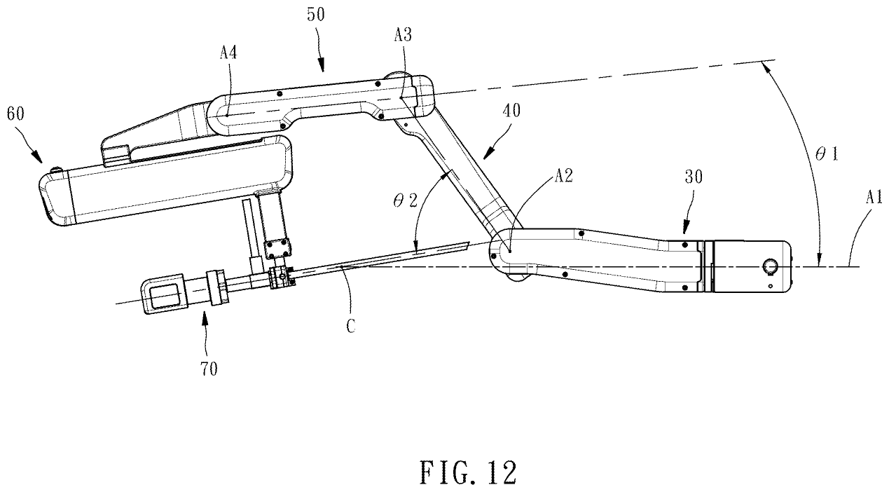

It can be seen from the above that when the first actuator 32 is actuated, the second transmission module 40 is driven by the first driven gear 35 to rotate around the second axial direction A2 relative to the first transmission module 30. During rotation of the second transmission module 40, the first planetary gear 42 is driven by the second transmission module 40 to rotate and revolve around the first fixed gear 37. At the same time, the third transmission module 50 is driven by the second driven gear 45 to rotate around the third axial direction A3 relative to the second transmission module 40. During rotation of the third transmission module 50, the second planetary gear 52 is driven by the third transmission module 50 to rotate and revolve around the second fixed gear 47. In addition, the fourth transmission module 60 is driven by the third transmission module 50 to rotate the fourth axial direction A4 relative to the third transmission module 50. During rotation of the fourth transmission module 60, the surgical instrument 70 is driven by the fourth transmission module 60 to rotate. However, no matter how the first, second, third, and fourth transmission modules 30, 40, 50, 60 move, as shown in FIGS. 11 and 12, the surgical instrument 70 is retained to revolve about a remote center of motion C (RCM).

On the other hand, the robotic arm 10 of the present invention further comprises a laser emitter 72. As shown in FIGS. 2 and 11, the laser emitter 72 is disposed in the first arm 31 for emitting a laser beam coaxial to the first axial direction A1 towards the remote center of motion C. As such, the surgical instrument 70 can be quickly positioned by means of the laser emitter 72 when set up. In addition, as shown in FIG. 6, the robotic arm 10 of the present invention further comprises a control unit 12 and an operation interface 14 (here, a plurality of buttons are taken as an example, but not limited to them). The control unit 12 is disposed in the third arm 51 and electrically connected with the first actuator 32, the second actuator 33, and the laser emitter 72. The operation interface 14 is disposed on the top surface of the third arm 51 for controlling startup and shutdown of the first actuator 32, the second actuator 33, and the laser emitter 72.

What needs to be added here is that FIG. 11 shows the robotic arm 10 of the present invention is in an initial state, and FIG. 12 shows the robotic arm 10 is in an extremely-stretched state. No matter how the first, second, third, and fourth transmission modules 30, 40, 50, 60 move, an angle θ 1 between the first transmission module 30 and the third transmission module 50 is between 4-8 degrees, and an angle θ2 between the surgical instrument 70 and the second transmission module 40 is between 62-66 degrees. If the aforesaid angle exceeds the range, the robotic arm 10 of the present invention cannot meet the conditions for clinical use, that is to say, the robotic arm 10 of the present invention can effectively meet requirements of pitch angle for clinical use. Further, as shown in FIG. 1, the first transmission module 30 and the third transmission module 50 are located at the same side of the second transmission module 40, so the overall volume can be reduced to facilitate the disassembly and assembly of components for maintenance. In addition, as shown in FIG. 11, the second axial direction A2 is offset upwards relative to the first axial direction A1, such that more space S can be provided below the first transmission module 30 to prevent the first transmission module 30 from collision with a patient's abdomen or other surgical instruments during operation.

As indicated above, the robotic arm 10 of the present invention is only equipped with a single first actuator 32 to achieve an effect of multi-arm linkage. During linkage movement, an effect of increasing the range of movement and improving the stability of the action can be achieved by means of cooperation between the first planetary gear 42 and the first fixed gear 37, and cooperation between the second planetary gear 52 and the second fixed gear 47. In addition, an effect of reducing overall volume can be achieved by setting the first transmission module 30 and the third transmission module 50 to the same side of the second transmission module 40.

What is claimed is:

1. A robotic arm for retaining a surgical instrument to revolve about a remote center of motion, the robotic arm comprising:
a fixed base;
a first transmission module including a first arm having a rear end thereof rotatably connected with the fixed base, a first actuator disposed in the first arm, a first driving gear disposed in the first arm and connected to the first actuator, a first driven gear rotatably connected to a front end of the first arm and engaged with the first driving gear, a first fixed shaft penetrated through the front end of the first arm and the first driven gear, and having one end thereof fixed to the first arm and the other end thereof protruding out of the first arm and connected to a first fixed gear;
a second transmission module including a second arm having a bottom end thereof receiving the first fixed gear and connected with the first driven gear, a first planetary gear rotatably connected with the bottom end of the second arm and engaged with the first fixed gear, a first transmission shaft disposed in the second arm and having two ends thereof connected with the first planetary gear and a second driving gear, and a second driven gear rotatably connected with a top end of the second arm and engaged with the second driving gear; and
a third transmission module connected with the second driven gear and the surgical instrument.

2. The robotic arm as claimed in claim 1, wherein the second transmission module further includes a second fixed shaft penetrated through the top end of the second arm and the second driven gear, and having one end thereof fixed to the second arm and the other end thereof protruding out of the second arm and connected with a second fixed gear; the third transmission module includes a third arm having a rear end thereof receiving the second fixed gear and connected with the second driven gear, a second planetary gear rotatably connected with the rear end of the third arm and engaged with the second fixed gear, a second transmission shaft disposed in the third arm and having two ends thereof connected with the second planetary gear and a third driving gear, and a third driven gear rotatably connected with a front end of the third arm and connected with the surgical instrument and engaged with the third driving gear.

3. The robotic arm as claimed in claim 1, further comprising a laser emitter disposed in the first arm for emitting a laser beam towards the remote center of motion.

4. The robotic arm as claimed in claim 3, wherein the first transmission module is rotatable relative to the fixed base around a first axial direction coaxial to the laser beam.

5. The robotic arm as claimed in claim 1, wherein an angle between the first transmission module and the third transmission module is between 4-8 degrees; an angle between the surgical instrument and the second transmission module is between 62-66 degrees.

6. The robotic arm as claimed in claim 2, wherein a gear ratio between the first fixed gear and the first planetary gear is the same as a gear ratio between the second driving gear and the second driven gear; a gear ratio between the second fixed gear and the second planetary gear is the same as a gear ratio between the third driving gear and the third driven gear.

7. The robotic arm as claimed in claim 1, wherein the first transmission module and the third transmission module are located at the same side of the second transmission module.

8. The robotic arm as claimed in claim 1, wherein the first fixed shaft has a first wireway coaxially communicating with a first wire hole of the first fixed gear; the second fixed shaft has a second wireway coaxially communicating with a second wire hole of the second fixed gear.

9. The robotic arm as claimed in claim 8, wherein the front end of the first arm has a first bearing portion and a first threaded hole provided at the first bearing portion;
the first fixed shaft has a first lower positioning hole radially communicating with the first wireway; the first transmission module further includes a first positioning plate pressing against the first bearing portion of the first arm and the first fixed shaft and having a first upper positioning hole, and a first bolt passing through the first upper and lower positioning holes and screwed to the first threaded hole; the top end of the second arm has a second bearing portion and a second threaded hole provided at the second bearing portion; the second fixed shaft has a second lower positioning hole radially communicating with the second wireway; the second transmission module further includes a second positioning plate pressing against the second bearing portion of the second arm and the second fixed shaft and having a second upper positioning hole, and a second bolt passing through the second upper and lower positioning holes and screwed to the second threaded hole.

10. The robotic arm as claimed in claim 4, wherein the second transmission module is rotatable relative to the first transmission module around a second axial direction offset upwards relative to the first axial direction.
